(19) United States
(12) Patent Application Publication
(10) Pub. No.: US 2011/0160668 A1
(43) Pub. Date: Jun. 30, 2011

(54) INFUSION PUMP MODULE

(75) Inventors: Yasunori SUGIMOTO, Kanagawa (JP); Teruki Kamada, Kanagawa (JP); Takashi Tomoyama, Kanagawa (JP)

(73) Assignee: RICOH COMPANY, LTD.

(21) Appl. No.: 12/978,409

(22) Filed: Dec. 24, 2010

(30) Foreign Application Priority Data
Dec. 28, 2009 (JP) 2009-298672
Dec. 15, 2010 (JP) 2010-279014

(51) Int. Cl. A61M 5/142

(52) U.S. Cl. 604/151

(57) ABSTRACT

An infusion pump module configured to transfer liquid includes a liquid inlet part including an inlet for the liquid; a liquid outlet part including an outlet for the liquid; a pump part including a piezoelectric element; and a flow sensor part configured to measure the flow rate of the liquid flowing through the infusion pump module, wherein the liquid inlet part, the liquid outlet part, the pump part, and the flow sensor part are connected to allow the liquid flowing in through the liquid inlet part to flow out through the liquid outlet part, and the liquid inlet part, the liquid outlet part, the pump part, and the flow sensor part are connected to form a single channel.

### CROSS-REFERENCE TO RELATED APPLICATIONS

The present application is based upon and claims the benefit of priority of Japanese Patent Application No. 2009-298672, filed on Dec. 28, 2009, and Japanese Patent Application No. 2010-279014, filed on Dec. 15, 2010, the entire contents of which are incorporated herein by reference.

### BACKGROUND OF THE INVENTION

1. Field of the Invention

The present invention relates to the technology of an infusion pump module including a flow sensor configured to measure the flow rate of liquid to be transferred.

2. Description of the Related Art

As a medical infusion pump module, a device is known that infuses a set amount by sandwiching a highly-elastic, self-expandable infusion tube in a machine and compressing the tube with rollers.

On the other hand, in the case of transferring a liquid medicine in a medical field where the medical liquid is injected into a patient, a liquid transfer pump may be used that includes a pump using a piezoelectric element and an oscillator circuit for driving the piezoelectric element. (See, for example, Japanese Laid-Open Patent Application No. 8-303352.) In such a liquid transfer pump, the piezoelectric element is caused to oscillate by applying a voltage of a predetermined frequency to the piezoelectric element, and with this oscillation, the medical liquid inside a channel is pushed out and transferred.

Further, the amount of liquid transferred may be controlled by installing a flow sensor in a liquid flow tube such as a tube connected to the liquid transfer pump and controlling the oscillator circuit in accordance with the measurement result of the flow sensor.

### SUMMARY OF THE INVENTION

According to one aspect of the present invention, an infusion pump module configured to transfer a liquid includes a liquid inlet part including an inlet for the liquid; a liquid outlet part including an outlet for the liquid; a pump part including a piezoelectric element; and a flow sensor part configured to measure a flow rate of the liquid flowing through the infusion pump module, wherein the liquid inlet part, the liquid outlet part, the pump part, and the flow sensor part are connected to allow the liquid flowing in through the liquid inlet part to flow out through the liquid outlet part, and the liquid inlet part, the liquid outlet part, the pump part, and the flow sensor part are connected to form a single channel.

The object and advantages of the embodiments will be realized and attained by means of the elements and combinations particularly pointed out in the claims.

It is to be understood that both the foregoing general description and the following detailed description are exemplary and explanatory and not restrictive of the invention as claimed.

### BRIEF DESCRIPTION OF THE DRAWINGS

Other objects, features and advantages of the present invention will become more apparent from the following detailed description when read in conjunction with the accompanying drawings, in which:

FIG. 1 is a diagram illustrating an infusion pump module according to a first embodiment;

FIG. 2 is a diagram illustrating a component configuration of the infusion pump module according to the first embodiment;

FIG. 3 is a diagram illustrating an infusion pump module according to a second embodiment;

FIG. 4 is a diagram illustrating a component configuration of the infusion pump module according to the second embodiment; and

FIG. 5 is a diagram illustrating a configuration of a system using the infusion pump module according to the first or second embodiment.

### DETAILED DESCRIPTION OF THE PREFERRED EMBODIMENTS

According to the above-described technique, however, the liquid transfer pump, the liquid flow tube, and the flow sensor installed in the liquid flow tube have to be prepared separately and connected to construct a system. Therefore, there is a problem in that the system increases in size to restrict its installation location.

According to one aspect of the present invention, an infusion pump module capable of controlling the amount of liquid transferred is provided that is compact in configuration to be free of restrictions on its installation location.

A description is given, with reference to the accompanying drawings, of embodiments of the present invention.

### [a] First Embodiment

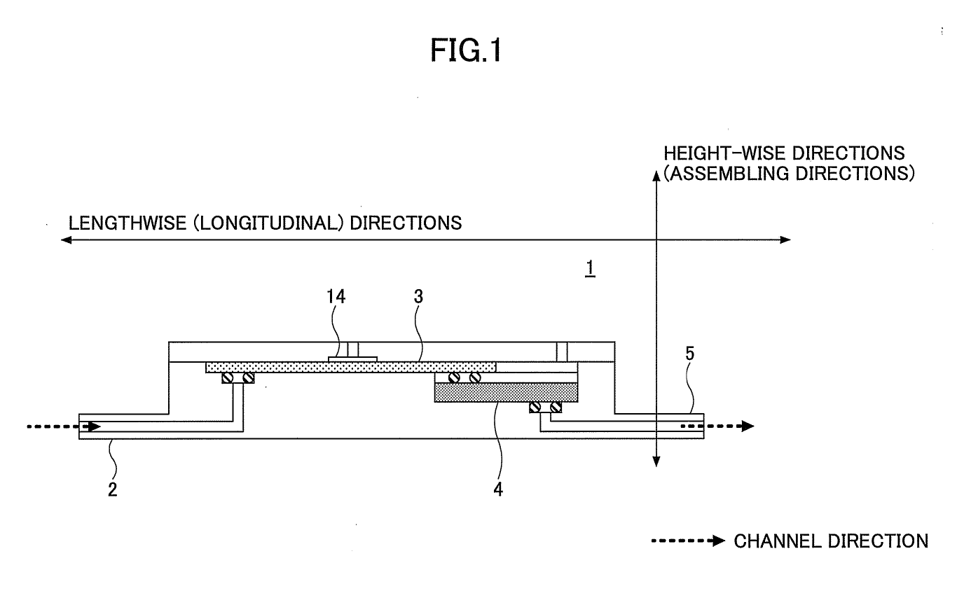

A description is given, with reference to FIG. 1, of a channel configuration of an infusion pump module 1 according to a first embodiment.

FIG. 1 is a diagram illustrating a configuration of the infusion pump module 1 according to the first embodiment. As illustrated in FIG. 1, the infusion pump module 1 includes a liquid inlet part 2, a pump 3, a flow sensor 4, and a liquid outlet part 5. In the infusion pump module 1, liquid flowing in through the liquid inlet part 2 is transferred to the flow sensor 4 via the pump 3 to flow out through the liquid outlet part 5. Here, the pump 3 includes a piezoelectric element 14. The pump 3 causes the piezoelectric element 14 to oscillate by applying a voltage of a predetermined frequency to the piezoelectric element 14, thereby implementing a pump function.

As illustrated in FIG. 1, according to the channel configuration of the infusion pump module 1, a single channel is formed by disposing the pump 3 and the flow sensor 4 parallel to each other so that the pump 3 and the flow sensor 4 at least partially face each other (there is at least an overlap between the pump 3 and the flow sensor 4 as viewed from above). Further, by adopting such a configuration, the overall length of the infusion pump module 1 may be reduced. In FIG. 1, the pump 3 and the flow sensor 4 are connected in this order in the liquid flow (channel) direction. Alternatively, the flow sensor 4 and the pump 3 may be connected in this order in the liquid flow direction.

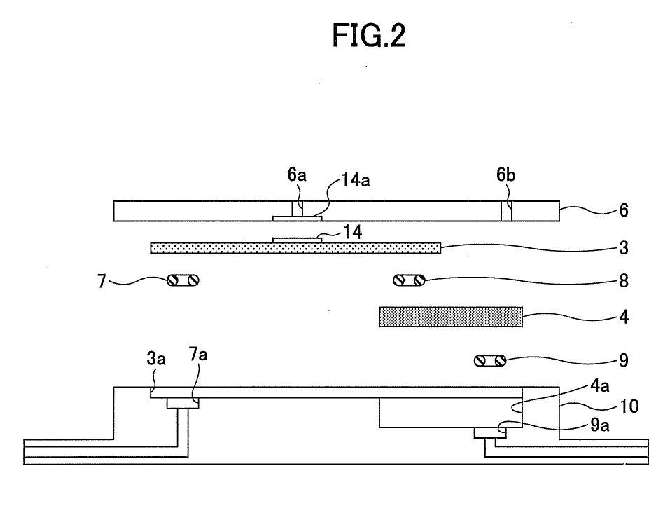

Next, a description is given, with reference to FIG. 2, of a component configuration of the infusion pump module 1.

FIG. 2 is a diagram illustrating components of the infusion pump module 1. As illustrated in FIG. 2, the infusion pump module 1 includes cases 6 and 10, the pump 3 (including the piezoelectric element 14), gaskets 7, 8, and 9, and the flow sensor 4.

At the time of assembling the infusion pump module 1, the cases 6 and 10 are fastened to each other so that the pump 3, the flow sensor 4, and the gaskets 7 through 9 are fixed at their respective predetermined positions. The case 6 and the case 10 are fastened to have a rectangular parallelepiped shape as a whole (except for the liquid inlet part 2 and the liquid outlet part 5).

A recess 14a for fitting in the piezoelectric element 14 is formed in the case 6.

In addition to a channel, a recess 3a for incorporating the pump 3, a recess 4a for incorporating the flow sensor 4, and recesses 7a and 9a for fitting in the gaskets 7 and 9 are formed in the case 10.

Further, the cases 6 and 10 may be waterproofed.

The pump 3 includes a liquid chamber (not graphically illustrated) for conveying liquid and the piezoelectric element 14 configured to generate power for conveying the liquid. Electric power is obtained from a probe of an external apparatus (not graphically illustrated) via a hole 6a provided in the case 6, and the piezoelectric element 14 is driven with this electric power to cause the liquid chamber to oscillate, thereby conveying the liquid.

The gaskets 8 and 9, which are O-rings, are configured to ensure airtightness and liquid-tightness. In particular, a soft (flexible) material is preferable for the gaskets 8 and 9. When the pump 3, the flow sensor 4, and the gaskets (O-rings) 7, 8, and 9 are incorporated and the cases 6 and 10 are fastened in the height-wise (assembling) directions as illustrated in FIG. 1, the gaskets 8 and 9 are moderately crushed (pressed) by the cases 6 and 10 and the flow sensor 4 to deform to connect the channels of the case 10, the pump 3, and the flow meter 4, thereby preventing the leakage of the liquid from between these components.

The flow sensor 4 is configured to measure the flow rate of the liquid conveyed (transferred) by the pump 3 and to transmit information on the flow rate to the external apparatus via a hole 6b provided in the case 6.

### [b] Second Embodiment

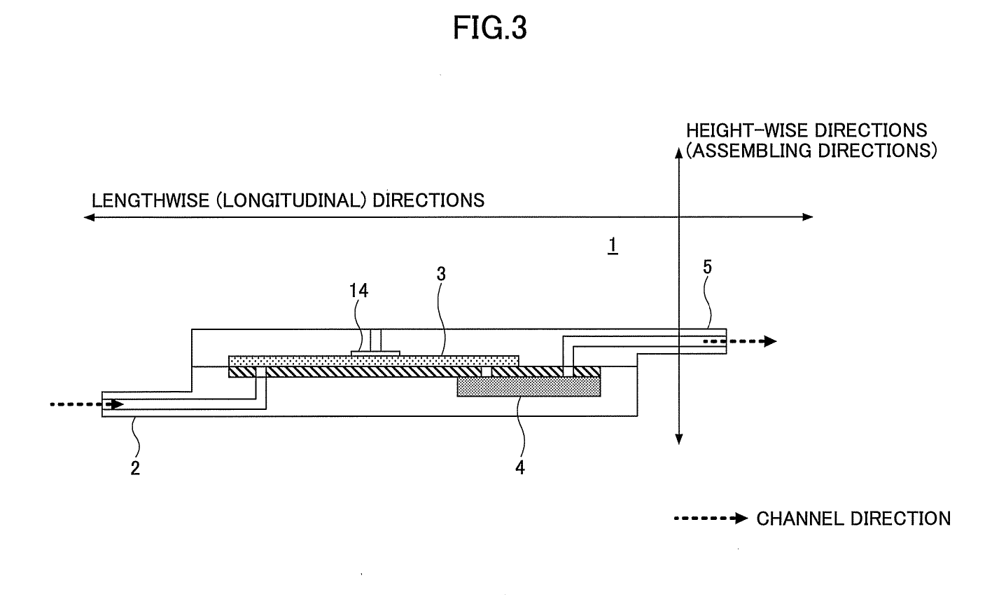

A description is given, with reference to FIG. 3, of a channel configuration of the infusion pump module 1 according to a second embodiment.

FIG. 3 is a diagram illustrating a configuration of the infusion pump module 1 according to the second embodiment. As illustrated in FIG. 3, the infusion pump module 1 includes the liquid inlet part 2, the pump 3, the flow sensor 4, and the liquid outlet part 5. In the infusion pump module 1, liquid flowing in through the liquid inlet part 2 is transferred to the flow sensor 4 via the pump 3 to flow out through the liquid outlet part 5. Here, the pump 3 includes the piezoelectric element 14. The pump 3 causes the piezoelectric element 14 to oscillate by applying a voltage of a predetermined frequency to the piezoelectric element 14, thereby implementing a pump function.

As illustrated in FIG. 3, according to the channel configuration of the infusion pump module 1, a channel is formed with a single structure by disposing the pump 3 and the flow sensor 4 parallel to each other so that the pump 3 and the flow sensor 4 at least partially face each other (there is at least an overlap between the pump 3 and the flow sensor 4 as viewed from above). Further, by adopting such a configuration, the overall length of the infusion pump module 1 may be reduced. In FIG. 3, the pump 3 and the flow sensor 4 are connected in this order in the liquid flow (channel) direction. Alternatively, the flow sensor 4 and the pump 3 may be connected in this order in the liquid flow direction.

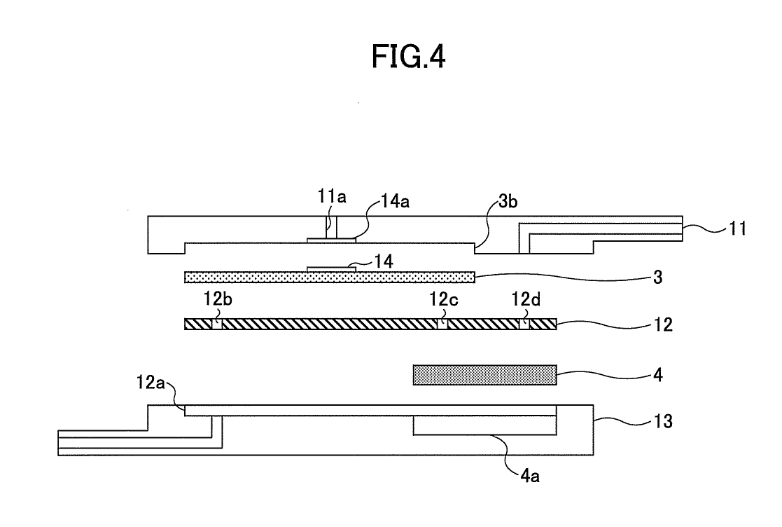

Next, a description is given, with reference to FIG. 4, of a component configuration of the infusion pump module 1.

FIG. 4 is a diagram illustrating components of the infusion pump module 1. As illustrated in FIG. 4, the infusion pump module 1 includes cases 11 and 13, the pump 3 (including the piezoelectric element 14), a gasket 12, and the flow sensor 4.

At the time of assembling the infusion pump module 1, the cases 11 and 13 are fastened to each other so that the pump 3, the flow sensor 4, and the gasket 12 are fixed at their respective predetermined positions. The case 11 and the case 13 are fastened to have a rectangular parallelepiped shape as a whole (except for the liquid inlet part 2 and the liquid outlet part 5).

In addition to a channel, a recess 3b for incorporating the pump 3 and the recess 14a for fitting in the piezoelectric element 14 are formed in the case 11.

In addition to a channel, a recess 12a for incorporating the gasket 12 and the recess 4a for incorporating the flow sensor 4 are formed in the case 13.

Further, the cases 11 and 13 may be waterproofed.

The pump 3 includes a liquid chamber (not graphically illustrated) for conveying liquid and the piezoelectric element 14 configured to generate power for conveying the liquid. Electric power is obtained from a probe of an external apparatus (not graphically illustrated) via a hole 11a provided in the case 11, and the piezoelectric element 14 is driven with this electric power to cause the liquid chamber to oscillate, thereby conveying the liquid.

The gasket 12, which is a soft (flexible) sheet for ensuring airtightness and liquid-tightness, is preferably a silicone rubber sheet. A hole 12b for introducing liquid from the liquid inlet part 2 to the pump 3, a hole 12c for transferring liquid from the pump 3 to the flow sensor 4, and a hole 12d for causing liquid from the flow sensor 4 to flow to the liquid outlet part 5 are formed in the gasket 12. The hole 12c of the gasket 12 serves as a connecting channel part configured to connect the pump 3 and the flow sensor 4.

Further, the connection of the pump 3 and the connecting channel part 12 and the connection of the flow sensor 4 and the connecting channel part 12 are aligned (coaxially positioned) in the height-wise (assembling) directions. This makes it possible to shorten the channel from the pump 3 to the flow sensor 4 and to reduce channel resistance, so that it is possible to simplify the infusion pump module 1. Further, it is possible to prevent a leakage of liquid by interposing a gasket of a single silicone rubber sheet between the pump 3 and the flow sensor 4. Therefore, it is possible to reduce the number of components.

As illustrated in FIG. 4, when the pump 3, the flow sensor 4, and the gasket 12 are incorporated and the cases 11 and 13 are fastened in the height-wise (assembling) directions, the gasket 12 is moderately crushed (pressed) by the cases 11 and 13 and the flow sensor 4 to deform to connect the channels of the cases 11 and 13, the pump 3, and the flow sensor 4, thereby preventing a leakage of liquid from between these components.

The flow sensor 4 is configured to measure the flow rate of the liquid conveyed (transferred) by the pump 3 and to transmit information on the flow rate to the external apparatus via a hole (not graphically illustrated) provided in the case 11.

### [Method of Using the Infusion Pump Module]

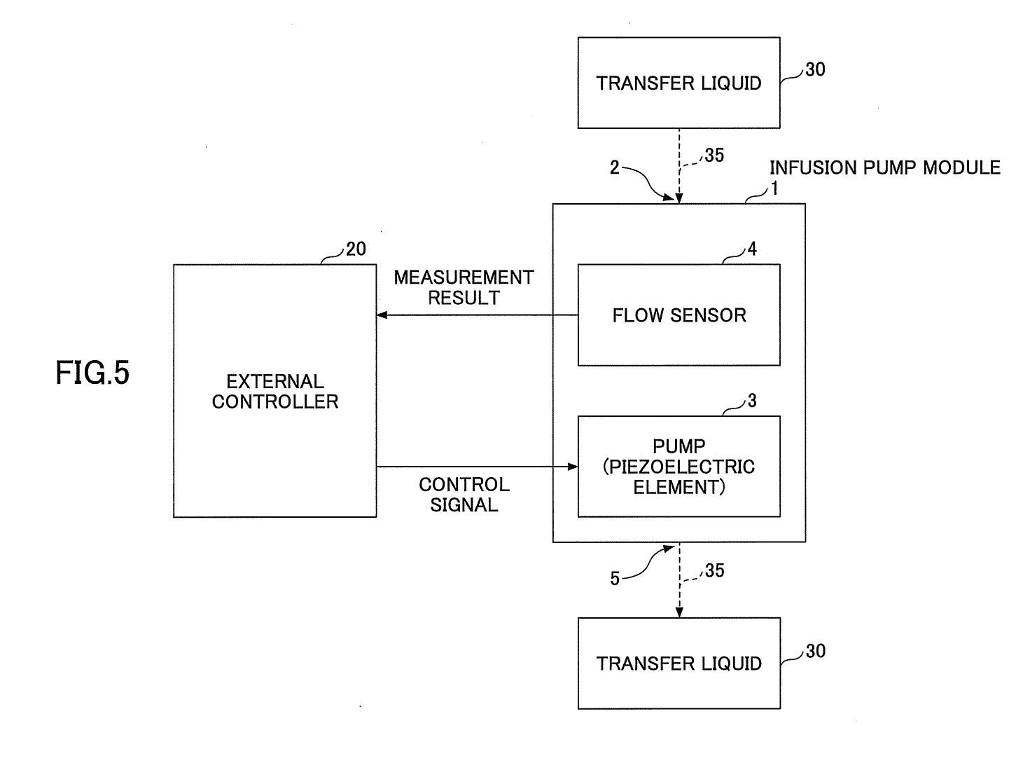

Next, a description is given, with reference to FIG. 5, of a method of using the infusion pump module 1.

FIG. 5 is a block diagram illustrating a system configuration of the infusion pump module including an external controller 20. As illustrated in FIG. 5, the infusion pump module 1 transfers transfer liquid (liquid to be transferred) 30 via a liquid transfer tube 35 connected to the liquid inlet part 2 and the liquid outlet part 5. Further, in this system, the infusion pump module 1 and the external controller 20 are connected, so that the external controller 20 is notified of the result of the measurement of the flow rate of the transfer liquid 30 by the flow sensor 4.

The external controller 20 generates a predetermined control signal based on the obtained result of the measurement of the flow rate, and transmits the generated predetermined control signal to the pump 3 (the piezoelectric element 14). Then, the piezoelectric element 14 of the pump 3 oscillates in accordance with the predetermined control signal, so that the pump 3 exerts a pump function for transferring the transfer liquid 30. Here, the relationship between the obtained result of the measurement of the flow rate and the predetermined control signal generated in accordance with the obtained result may be fittingly adjusted in the external controller 20.

According to the above-described infusion pump modules of the first and second embodiments, for example, the following effects may be produced.

There is no leakage of liquid because one or more soft (flexible) gaskets such as an O-ring and a silicone rubber sheet are used to prevent a leakage of liquid.

The disclosed infusion pump modules use one or more gaskets, so that there is no run-out of an adhesive agent or a solvent. Further, the disclosed infusion pump modules may be disposable. Therefore, the disclosed infusion pump modules require no cleaning, thus being safe without a medical residue or germs entry.

Further, the disclosed infusion pump modules are capable of maintaining a set flow rate by containing a flow sensor and performing feedback control, thus having good controllability.

Further, the disclosed liquid pump modules use a small-size pump using a piezoelectric element and a small-size flow sensor, and may therefore be reduced in size.

Further, the disclosed infusion pump modules separate low-cost, irreplaceable components and reusable components, and provide a part to connect them. Further, the components are inexpensive, and the structure is simple. Therefore, the quality is easy to stabilize, and the assembly cost is low.

According to one aspect of the present invention, an infusion pump module is capable of controlling the amount of liquid transferred, and is compact in configuration to be free of restrictions on its installation location.

All examples and conditional language recited herein are intended for pedagogical purposes to aid the reader in understanding the invention and the concepts contributed by the inventors to furthering the art, and are to be construed as being without limitation to such specifically recited examples and conditions, nor does the organization of such examples in the specification relate to a showing of the superiority or inferiority of the invention. Although the embodiments of the present invention have been described in detail, it should be understood that various changes, substitutions, and alterations could be made hereto without departing from the spirit and scope of the invention.

What is claimed is:

1. An infusion pump module configured to transfer a liquid, comprising:
a liquid inlet part including an inlet for the liquid;
a liquid outlet part including an outlet for the liquid;
a pump part including a piezoelectric element; and
a flow sensor part configured to measure a flow rate of the liquid flowing through the infusion pump module,
wherein the liquid inlet part, the liquid outlet part, the pump part, and the flow sensor part are connected to allow the liquid flowing in through the liquid inlet part to flow out through the liquid outlet part, and
the liquid inlet part, the liquid outlet part, the pump part, and the flow sensor part are connected to form a single channel.

2. The infusion pump module as claimed in claim 1, wherein the pump part and the flow sensor part are disposed to overlap each other in a longitudinal direction of the infusion pump module.

3. The infusion pump module as claimed in claim 2, wherein the pump part and the flow sensor part are parallel to each other in the longitudinal direction of the infusion pump module.

4. The infusion pump module as claimed in claim 3, further comprising:
a connecting channel part configured to connect the pump part and the flow sensor part,
wherein at connections of the liquid inlet part, the liquid outlet part, the pump part, the flow sensor part, and the connecting channel part, connected states are maintained with forces in assembling directions of the infusion pump module.

5. The infusion pump module as claimed in claim 4, wherein the connection of the pump part and the connecting channel part and the connection of the flow sensor part and the connecting channel part are coaxially positioned in the assembling directions.

6. The infusion pump module as claimed in claim 5, wherein O-rings are used at the connections of the liquid inlet part, the liquid outlet part, the pump part, the flow sensor part, and the connecting channel part.

7. The infusion pump module as claimed in claim 4, wherein the connecting channel part comprises a flexible gasket interposed between the pump part and the flow sensor part.
